(19) United States
(12) Patent Application Publication
(10) Pub. No.: US 2009/0082784 A1
(43) Pub. Date: Mar. 26, 2009

(54) Interventional medical system

(75) Inventors: Oliver Meissner, Munchen (DE); Simone Prummer, Neunkirchen am Brand (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/283,410

(22) Filed: Sep. 11, 2008

(30) Foreign Application Priority Data
Sep. 21, 2007 (DE) 10 2007 045 075.5

(51) Int. Cl. A61B 19/00

(52) U.S. Cl. 606/130

(57) ABSTRACT

The invention relates to an interventional medical diagnosis or therapy system comprising: an imaging device, which is embodied for recording 3D images of an object under examination, with a control unit and an image processing unit; an at least two-axis robot arm, having means for yield control of the robot arm and a robot control unit for control of the robot arm; an instrument holder, which is connected to the robot arm, for holding an instrument; a position detection device, by means of which the position of the instrument is able to be detected for the robot control unit. The robot arm is embodied to yield to system-external force effects or to follow a system-external guidance.

### CROSS REFERENCE TO RELATED APPLICATIONS

This application claims priority of German application No. 10 2007 045 075.5 filed Sep. 21, 2007, which is incorporated by reference herein in its entirety.

### FIELD OF THE INVENTION

The invention relates to an interventional medical diagnosis or therapy system.

### BACKGROUND OF THE INVENTION

Interventional procedures, as employed in radiology for example, already have an important role to play in the diagnosis and therapy of many illnesses. One variant in such cases uses rigid instruments, e.g. needles, to reach a specific point in the body and within an organ from outside through the skin (percutaneously). This is used in puncturing, biopsies, ablations, brachytherapies and many other interventional procedures.

As a result of further technical development of imaging modalities such as computer tomography, magnetic resonance tomography or angiography for example, inflammatory or tumorous changes can be detected at an ever earlier stage. In addition ever finer instruments are being developed for puncturing, catheterization and probing the organ systems. To subject the patient to as little stress as possible minimally-invasive interventions are being used every more frequently. Because of the fact that ever smaller target areas of the body can be accessed, the requirements for more precise and well-targeted puncturing methods are also increasing.

Since the doctor cannot directly see the instrument in the patient's body, he relies on the support of imaging methods. Ideally a 3D data set of an imaging modality (e.g. magnetic resonance tomograph, computer tomograph, angiography device) is available to the doctor prior to the intervention, in which he can see the target region and, based on the physiology, can plan the ideal path and thus the entry point of the instrument.

In a far simpler variant the target region is scanned during the intervention for example by means of a C-arm x-ray device which can generate 3D images, and the further planning is undertaken on the basis of the combined information from 2D and 3D data. The puncture point in the body and the orientation of the needle are defined with this planning. This planning can then be passed on to a navigation system which is registered with the 3D data record, and adjusted for aligning the needle by means of various manual methods. The advance of the needle can be monitored in such cases fluoroscopically, i.e. in real time using x-rays or by means of ultrasound. In another known method the pre-interventional 3D data set is registered with the C-arm data set and the information is subsequently used for navigation.

In general planning of the needle channel is conducted by a doctor by means of a therapy planning system, i.e. virtually with the aid of software suitable for the purpose and subsequently transferred manually to the instrument.

Problems emerge for example with obese or powerful patients. Because of the technical implementation of the C-arm x-ray device only a restricted body volume can be reconstructed. It thus occurs that the surface of the body is not contained in the 3D data set. In virtual planning the doctor does not recognize in this case where the puncturing point lies and whether the planned access path is unsuitable for the intervention for example because of the overlaying of bones or ribs.

At present for example a fine needle biopsy or a thermoablation of a seat of an illness in the liver is mostly conducted percutaneously under CT control. In this case the doctor performing the intervention uses for his access planning a combination of the CT sectional image, external markers applied to the surface of the patient's skin and also the orientation aid provided by a laser cross-hair mounted on the CT gantry. The actual puncturing as well as the movement of the puncturing needle is undertaken in this method essentially on the basis of the subjective control by the doctor. Depending on the level of experience of the examining physician multiple puncturing is necessary in such cases. This process, in addition to being uncomfortable for patients, brings with it an increased risk of complications, such as bleeding, damage to organs or hematomas. In addition the puncturing accuracy is restricted with this process, especially with small target regions.

In order to achieve a targeted instrument guidance, various other supporting navigation aids can be used nowadays, for example optical or electromagnetic positioning systems or the use of a stereotactic frame, of which the position in the room is known and which has a facility for instrument guidance. However this is a complicated method of finding the insertion point and aligning the needle exactly. On the other hand the distal end of the needle remote from the patient is monitored by the position finding system. Thus possible deformations of the needle tip triggered by the body's resistance and which, under some circumstances, can lead to a deviation from the puncturing path, are not taken into account.

### SUMMARY OF THE INVENTION

The object of the present invention is to provide an interventional diagnosis and/or therapy system which allows especially simple, intuitive and safe interventional actions.

The object is inventively achieved by an interventional medical diagnosis and/or therapy system with an at least two-axis robot arm in accordance with the independent claim. Advantageous embodiments of the invention are the subject matter of the dependent claims in each case.

The central idea behind the invention lies in providing an interventional medical diagnosis and/or therapy system which features an at least two-axis robot arm with an instrument holder attached thereto for holding an instrument and which features a yield control, with the robot arm being embodied to yield to force effects external to the system and/or to follow system-external guidance. By gripping and moving the structural parts, the robot arm, especially a lightweight robot, and thus the instrument holder with the instrument arranged on it, can be intuitively positioned as required by the user.

This enables a user to position the instrument or make positional corrections quickly and easily, without having to operate a complex control program. Such a system makes possible an intuitive method to assist in the planning and execution of puncturings or biopsies for example. Such a yield control can for example be implemented by means of a number of sensors, such as position sensors and force sensors, which are arranged on the individual joints of the lightweight robot. In particular this type of yield control is known for a lightweight robot made by KUKA Roboter GmbH.

The inventive interventional medical diagnosis and/or therapy system also features an imaging device with a control unit and an image processing unit and a position detection facility, by means of which a robot control unit detects the position of the instrument. The imaging device is used for creating a 3D data set of the area of a patient's body to be examined/undergo the therapy, so that the target area can be localized especially precisely. The position detection facility allows a positioning of the instrument such that the position of the instrument tip is known to the robot control unit. This makes an especially safe control of the instrument possible.

In accordance with an embodiment of the invention the robot arm has a first mode of operation in which the robot arm yields to system-external force effects and/or follows a system-external guidance, and a second mode of operation in which the robot arm is fixed in relation to system-external force effects, with the robot arm being switchable between the first and the second mode of operation. Thus for example the first mode of operation can be activated to set the position of the instrument at the point of entry, so that the advantages of the yield control can be exploited while the doctor is intuitively positioning the instrument. Subsequently a switch is made into the second mode of operation in which the instrument can no longer be adjusted manually by the doctor, in order to suppress a change in position caused by inadvertent movements. Further modes of operation can also be provided in which for example a yield is only provided in specific directions and along specific paths.

According to a further embodiment of the invention the position detection facility for detecting the position of the instrument and especially of its tip can be embodied by a positioning system with an evaluation unit. In an advantageous manner the positioning system for finding the position of the instrument, especially of the instrument tip, operates on the basis of optical or electromagnetic position finding. Optical markings can be arranged for this purpose on the instrument holder or the instrument for example which, together with a tracking system, make the location and the orientation of the instrument detectable. A laser can also be arranged on the imaging device for example, which records the position of the instrument by triangulation. Furthermore coils can also be arranged on the instrument holder or the instrument which, together with an electromagnetic navigation system, display the position of the instrument.

The position information is subsequently passed on to the robot control unit and/or to the control unit of the imaging device. In the simplest case the position detection facility consists of a fixed connection between the instrument holder and the robot arm, in a permanently-defined relative position, so that the position of the instrument or of its tip is always able to be calculated even with movement of the robot arm in relation to the relative position.

In an advantageous manner the diagnosis and/or therapy system features a device for registration of the robot arm with the imaging device. This registration is advantageous to enable a relative position between the target area and the instrument to be determined. The device for registration of the robot arm with the imaging device can for example be embodied by a defined mechanical coupling or a rail with position markers between the robot arm and the imaging device.

According to a further embodiment of the invention the diagnosis and/or therapy system feature a therapy planning system which is connected to the imaging device and the robot control unit of the robot arm for example for data exchange. These types of therapy planning systems can for example be embodied by planning software which can be operated on a conventional PC.

In an advantageous manner the imaging device is embodied by a C-arm x-ray device. Such a C-arm x-ray device allows the doctor particularly good access to the patient. In addition this type of C-arm x-ray device can easily record tomographic images, which can subsequently be reconstructed to form a three-dimensional volume image.

In a further embodiment the robot arm can also be combined with an imaging device in the form of an ultrasound head. In this way a radiation-free positioning of the needle tip in real time under visual control is made possible.

According to a further embodiment of the invention the robot arm is formed by a lightweight robot with at least six, especially seven, degrees of freedom. The at least six degrees of freedom allow an especially high flexibility and ability to manipulate the lightweight robot. A lightweight robot in such cases is to be understood as an especially small and light robot arm. The lightweight robot has the advantage of being in a position to share its working area with people. To this end the lightweight robot is in particular smaller than a human being.

Expediently the diagnosis and/or therapy system possesses a movable patient table. An activation of the patient table can for example be integrated into the control and image processing unit of the imaging device. Such a patient table has the advantage of being able to be moved during an interventional access to support the doctor and for example guaranteeing better accessibility to the patient.

Expediently the diagnosis and/or therapy system features a further positioning system for finding the position of the instrument, especially of the instrument tip. In this case for example there can be provision for using two positioning systems, which are based on different measurement principles, in order to allow an especially precise positioning of the instrument tip.

### BRIEF DESCRIPTION OF THE DRAWINGS

The invention, as well as further advantageous embodiments in accordance with features of the subclaims, are explained in greater detail below with reference to schematic exemplary embodiments in the drawing, without the invention being thus restricted to these exemplary embodiments. The figures show:

FIG. 1 an overhead view of an inventive interventional diagnosis and therapy system with a lightweight robot for instrument guidance;

FIG. 2 a workflow for operation of the inventive interventional diagnosis and therapy system.

### DETAILED DESCRIPTION OF THE INVENTION

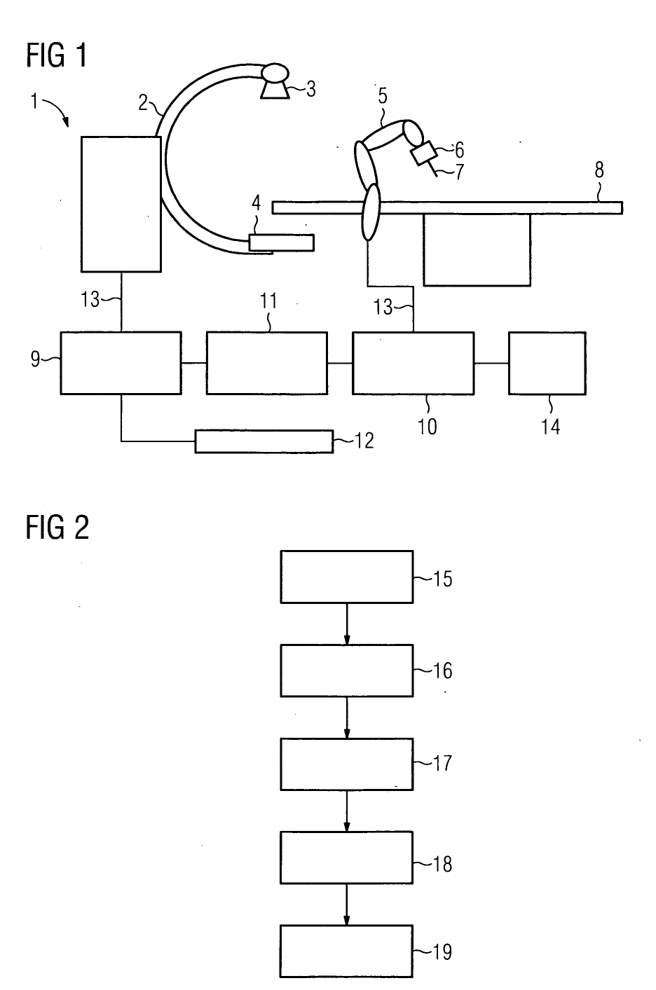

FIG. 1 shows an interventional diagnosis and/or therapy system 1 in accordance with the invention. The diagnosis and/or therapy system features an x-ray device with a C-arm 2, with an x-ray source 3 and an x-ray detector 4 being mounted on said C-arm 2. A control and image processing unit 9 is used for control of the x-ray device as well as for processing of the recorded image data. The diagnosis and/or therapy system also features a lightweight robot 5 which uses an instrument holder 6 to hold an instrument 7, for example a biopsy needle. The lightweight robot 5 is controlled by a robot control unit 10. In addition the system features a therapy planning system 11, a positioning system with an evaluation unit 14 and possesses a data connection to a network 12.

A lightweight robot 5 in this case is especially to be understood as a small and compact robot arm. A lightweight robot, just like a conventional industrial robot, can have a number of joints and degrees of freedom. The use of an at least six-axis articulated arm robot is especially advantageous, since this can carry out all possible three-dimensional movements.

The lightweight robot 5 is embodied such that it can both be controlled from the robot control unit 10 and also features means for yield control. Yield in this context is to be understood as the lightweight robot yielding to system-external force effects, i.e. allows the doctor to push it gently as well as allowing system-external guidance to be followed, i.e. it allows a doctor to correct its position by moving it. By gripping and moving parts of its structure the lightweight robot can thus be intuitively positioned as required by the user. This type of lightweight robot has been developed for example by KUKA Roboter GmbH.

A yield control, i.e. a setting of the yield of the lightweight robot, especially depending on its position, can for example be monitored by means of a number of sensors, such as position sensors and force sensors, which are arranged on the individual joints of the lightweight robot. The force sensors can be used to obtain fast and high-resolution local information about the objects with which the robot is in contact. To this end is for example each joint of the lightweight robot is equipped with a drive-side position sensor as well as drive side position and moment sensors, since the yield can be represented as a combination of position (orientation) and force (moment). This allows the lightweight robot to be operated under position, speed and force control. Movement paths can thus be followed, precisely, dynamically and without vibration.

A diagnostic-therapeutic workflow for operating the inventive interventional diagnosis or therapy system is shown in FIG. 2 and described below. Such a workflow is especially simple and intuitive:

a. Initially in a first step 15 a three-dimensional data set is recorded by means of the C-arm CT x-ray device, with the data set mapping the area of the body to be examined/undergo therapy and the precise target region (i.e. for example an organ or a lesion of body tissue) is defined in this 3D data set. Optionally the data set created by means of the C-arm CT x-ray device can also be used with another data set recorded prior to the intervention (for example ultrasound, CT, MR, PET, SPECT).

b. In a second step 16 an operator, for example a doctor, determines on the basis of the known localization, the insertion point on the surface of the patient's body, for example based on his or her medical experience and the recognizable anatomy (e.g. ribs), and positions the instrument held by means of the instrument holder of the robot arm, especially the lightweight robot, for example a puncturing or biopsy needle. To do this the operator controls the instrument holder with the fixed instrument manually by gripping the instrument holder and positioning it as per requirements. This is advantageously possible because the robot arm is embodied to yield to system-external force effects and to follow system-external guidance, by the doctor for example.

c. In a third step 17 the control unit of the lightweight robot aligns the instrument holder and thereby the instrument automatically on the target region, with the position of the instrument tip and thereby of the insertion point being maintained. Such an alignment of the instrument is possible in a simple manner through the registration of the robot arm with the imaging device. Before the orientation of the instrument provision can still be made for position information of the target region and the instrument tip to be transmitted to the robot control unit of the robot arm.

d. In a fourth step 18, in parallel to the third step, the virtual path of the instrument is shown in the therapy planning system, e.g. in a 3D visualization or alternatively in three planes in two-dimensional images. This enables the doctor to additionally check whether the virtual path is worthwhile. In the cases in which structures that it is critical to bypass, such as vessels, bones or organs, lie on the path, the operator moves the instrument or corrects the virtual path, subsequently the planning of the puncturing path is automatically or manually adapted in the therapy planning system. If required it is also possible to move the path or the virtual needle in the therapy planning system and the lightweight robot follows this movement.

e. In a fifth step 19, after confirmation, the lightweight robot moves to the predetermined position, e.g. by the second operating mode being set automatically. After enabling by the doctor, a switch is made to a further mode of operation, in which the fixing of the instrument is especially only enabled in the optimum direction for puncturing. The puncturing needle is moved manually by the doctor. The needle can be advanced under control, e.g. fluoroscopy or by means of an electromagnetic or optical positioning system.

Especially advantageously a multi-axis lightweight robot can move its arm (actively or through manual intervention) and thus fixes the position and orientation of the instrument holder. This is necessary if the arm of the lightweight robot is in the way of the C-arm or of the doctor performing the examination.

With the inventive interventional medical diagnosis and/or therapy system and the corresponding operation described above of said system, it is possible to hit even small lesions deep within regions of the body of a patient with an instrument attached to a robot arm, with operation and use being intuitive, simple and illuminating for the doctor.

In the conventional workflow all steps have still been actively conducted by the doctor, especially also the setting of path planning, the finding of the insertion point and the orientation of the instrument. The inventive, intuitive workflow on the other hand is summarized as follows:
Creation of a 3D data record of a patient with an medical imaging device (initiated by the doctor),
Determination of a target region of the patient to which the therapy is to be applied based on the 3D data set (manually by the doctor),
Planning an entry point of an instrument on the surface of the patient (also manually by the doctor, possibly supported by a therapy planning system),
Positioning the instrument tip at the point of insertion by manual movement of a robot arm which holds the instrument (the doctor moves the yield-controlled robot arm),
Transmission of the position information of the target region and of the instrument tip to a robot control unit of the robot arm,
Automatic orientation of the instrument by the robot control unit to the target region (with the position of the instrument tip being maintained),
Automatic checking of the path of the instrument by means of the therapy planning system,
Additional optional manual checking of the path of the instrument (by the doctor),
Manual movement of the instrument (by the doctor,
Optional checking during the advance of the instrument (e.g. through images generated by fluoroscopy or another imaging method).

The invention can be briefly summarized as follows: To undertake especially simple, intuitive and safe interventions an interventional medical diagnosis and/or therapy system is provided, comprising
an imaging device, which is embodied for recording 3D images of an object under examination, with a control unit and an image processing unit (9),
an at least two-axis robot arm, having I means for yield control of the robot arm and a robot control unit (10) for control of the robot arm,
An instrument holder (6), which is connected to the robot arm, for holding an instrument (7),
A position detection device, by means of which the position of the instrument (7) is able to be detected for the robot control unit (10),
   with the robot arm being embodied to yield to system-external force effects and/or to follow a system-external guidance.

1.-12. (canceled)

13. An interventional medical system, comprising:
an imaging device that records a 3D image of a patient;
an instrument holder that holds an instrument of the medical system;
a robot arm connected to the instrument holder that moves the instrument;
a robot control unit that controls the robot arm based on a position of the instrument; and
a position detection device that detects the position of the instrument,
wherein the robot arm is configured to yield to a force external to the medical system.

14. The interventional medical system as claimed in claim 13, wherein the robot arm follows a guidance external to the medical system.

15. The interventional medical system as claimed in claim 13, wherein the robot arm comprises a first mode of operation in which the robot arm yields to the force external to the medical system.

16. The interventional medical system as claimed in claim 15, wherein the robot arm comprises a second mode of operation in which the robot arm is fixed with respective to the force external to the medical system.

17. The interventional medical system as claimed in claim 16, wherein the robot arm is operated by switching between the first and the second mode of operation.

18. The interventional medical system as claimed in claim 13, wherein the robot arm is registered with the imaging device.

19. The interventional medical system as claimed in claim 13, wherein a therapy planning system is connected to the imaging device and the robot control unit.

20. The interventional medical system as claimed in claim 13, wherein the imaging device is a C-arm x-ray device.

21. The interventional medical system as claimed in claim 13, wherein the robot arm is a lightweight robot with at least six degrees of freedom.

22. The interventional medical system as claimed in claim 13, further comprising a movable patient table.

23. The interventional medical system as claimed in claim 13, wherein the position detection device is a part of a positioning system.

24. The interventional medical system as claimed in claim 23, wherein the positioning system is an optical or electromagnetic position system.

25. The interventional medical system as claimed in claim 23, wherein the position system determines an advance of the instrument.

26. The interventional medical system as claimed in claim 23, wherein the position system detects a tip of the instrument.

27. The interventional medical system as claimed in claim 13, wherein the interventional medical system is for a diagnosis or a therapy of the patient.

28. A method for performing an intervention of a patient, comprising:
creating a 3D data record of the patient by an medical imaging device;
determining a target region of the patient based on the 3D data record;
planning an insertion point of an instrument on a surface of the patient;
positioning a tip of the instrument at the insertion point by manual moving a robot arm holding the instrument;
transmitting a position information of the target region and the tip of the instrument to a robot control unit;
automatically positioning the instrument by moving the robot arm via the robot control unit on the target region;
automatically checking a path of the instrument by a therapy planning system;
manually advancing the instrument along the path in a direction of the target region; and
checking a position of the tip of the instrument during the advance of the instrument.

29. The method as claimed in claim 28, further comprising manually checking the path of the instrument.
